(19) United States
(12) Patent Application Publication
(10) Pub. No.: US 2023/0391978 A1
(43) Pub. Date: Dec. 7, 2023

(54) ENZYMATIC RECYCLING OF POLYURETHANES BY CUTINASES

(71) Applicant: SOCIETE DES PRODUITS NESTLE S.A., Vevey (CH)

(72) Inventors: TIM BORNER, Linwood, NJ (US); VANESSA SREBNY, VANESSA (DE); NINA CHRISTINA MARIA ROOTHANS, Singen (DE)

(21) Appl. No.: 18/250,066

(22) PCT Filed: Oct. 22, 2021
(86) PCT No.: PCT/EP2021/079325

§ 371 Date: Apr. 21, 2023

(30) Foreign Application Priority Data
Oct. 23, 2020 (EP) 20203524.2

(51) Int. Cl. C08J 11/10

(52) U.S. Cl. CPC C08J 11/105; C08J 2375/04

(57) ABSTRACT

The present invention relates generally to the field of degrading polyurethane (PU), for example PU layers in multi-layer packaging. For example, the present invention relates to a method of degrading polyurethane (PU) in packaging material comprising the step of subjecting the packaging material comprising the PU to at least one cutinase. The PU may be a PU-based layer in a multilayer packaging structure comprised in a packaging. Remarkably, the subject matter of the present invention allows the selective degradation of PU containing layers in multi-layer packaging materials.

### FIELD OF THE INVENTION

The present invention relates generally to the field of degrading polyurethane (PU), for example PU layers in multi-layer packaging. For example, the present invention relates to a method of degrading polyurethane (PU) in packaging material comprising the step of subjecting the packaging material comprising the PU to at least one cutinase. The PU may be a PU-based layer in a multilayer packaging structure comprised in a packaging. Remarkably, the subject matter of the present invention allows the selective degradation of PU containing layers in multi-layer packaging materials.

### BACKGROUND OF THE INVENTION

Plastic production has been increasing for over the last six decades, reaching 348 million tonnes in 2017 (Plastics Europe, 2018). Packaging is the major sector of plastic usage, with almost 40% of the market demand (Plastics Europe, 2018). It consists for a large part of single-use plastics, which have a short lifetime, turning to waste shortly after being acquired by the consumer. It is common knowledge that plastic accumulation is a current major environmental concern, resulting from the high resistance of plastics to degradation, together with improper disposal or deposition of waste in landfills. Yet, efforts have been made over the past years to avoid plastic deposition in landfills (Plastics Europe, 2018). Nevertheless, a large amount of packaging plastics still ends up as waste, so efficient recycling technologies are needed to simultaneously minimize the amount of produced waste and the resource consumption to produce plastics.

Polymers used in packaging can be divided into two main groups: the ones with a carbon-carbon backbone [e.g., polypropylene (PP), polyethylene (PE), polyvinyl chloride (PVC) and polystyrene (PS)] and those with a heteroatomic backbone [e.g., polyesters and polyurethanes (PU)]. The high energy required to break C—C bonds makes hydrocarbons very resistant to degradation (Microb Biotechnol, 10(6), 1308-1322). On the other hand, polyesters and polyurethanes have hydrolysable polyester bonds so they are less resilient to abiotic and biotic degradation.

The most common polyester is polyethylene terephthalate (PET) (Plastics Europe, 2018). Plastic packaging is usually not composed of one single polymer. Instead, blends or multiple layers of different polymers are often required to obtain certain properties (elasticity, hydrophilicity, durability or water and gas barrier) related to the specific application of the plastic (Process Biochemistry, 59, 58-64). Also, packaging materials generally contain adhesives, coatings and additives, such as plasticizers, stabilizers and colorants (Philos Trans R Soc Lond B Biol Sci, 364(1526), 2115-2126). This makes the recycling of some packaging materials very difficult.

Current plastic waste recycling technologies predominantly consist of thermo-mechanical processes, while chemical recycling is in its early industrialization phase. Mechanical recycling requires clean input waste streams that may be achieved through prior cleaning and separation steps in the case of contaminated and complex packaging structures, respectively. Thus, the recycling rates of multilayer packaging today are very low. Instead, multilayer packaging is mostly incinerated or ends up in landfills. Besides, the mechanical recycling process often results in downgraded plastics with decreased properties and limited food grade quality, thus losing their original value and application. These materials are then typically used for lower-value secondary products. On the other hand, chemical recycling processes are being developed to enable the recovery of the polymer's building blocks that can be used to remake the plastic. However, this process is economical and energetically costly and usually requires extreme conditions and harsh chemicals. These technologies are thus not ideal for complex, multilayer plastic materials (Process Biochemistry, 59, 58-64).

A technology enabling the selective removal and recycling of each component of multilayer plastic packaging would provide the possibility of reproducing the original packaging and expanding recycling to mixed plastic packaging waste and materials.

Enzymes are very selective towards their substrate, so they offer a high potential to be applied in recycling processes. Enzymes would enable the selective decomposition of each layer into either the starting building blocks, which can be used for subsequent production of new plastics or as added-value chemicals. The enzymatic and microbial degradation of recalcitrant plastics has been increasingly studied over the past years, with particular focus on PET (Microb Biotechnol, 10(6), 1302-1307). Even though the enzymatic degradation of plastic is difficult, there are enzymes capable of degrading polyesters used in the production of plastic packaging. The degradation efficiency of enzymes however varies with different classes and types of enzymes, and the conditions under which the experiments were carried out highly influence the extent of degradation. In addition, the polymer properties, e. g., crystallinity and composition, also have a strong influence on the rate of degradation.

Even though efforts have been made to increase the efficiency of enzymatic degradation of polymers, most studies were performed on pure materials. Although these studies provide a good initial insight on the enzymatic degradation of plastics, they are not representative of actual packaging materials as polymers are not isolated in this case and additives may be present. Moreover, a deep understanding of the effect of experimental conditions, enzyme properties and polymer properties on the degradation process is lacking.

Therefore, to design a selective recycling process for multi-layer packaging is of high importance.

Last but not least, degradation of PU by enzymatic reaction was described in the past, for instance from U.S. Pat. No. 6,255,451. However, this patent application discloses enzymatic degradation for polyurethanes that contain urea linkages to make them biodegradable as such, due to the fact that urea linkages are more prone to chemical and enzymatic hydrolysis as disclosed in U.S. Pat. No. 6,255,451. Thus, this document does not provide any solution for degradation of non-biodegradable polyurethanes. Moreover, U.S. Pat. No. 6,255,451 concerns the application of lipases and esterases on polyurethane types that are used in textile, leather aircraft [E. Windemuth, H. Gensel, M. Kramer. Melliand Textilber., 37 (1956), pp. 843-846; H. Traeubel. J. Am. Leather Chem. Assoc., 83 (9) (1988), pp. 317-327], but are not typically used in packaging applications, and does not provide any degradation data on cutinases.

It would therefore be desirable to have available a process that can be used to selectively degrade PU-based layers in multi-layer packaging that is cost efficient, results in high quality materials and does not require harsh processing conditions, and especially for degrading PU materials that are not been reported as biodegradable and are typically been used in packaging applications.

Any reference to prior art documents in this specification is not to be considered an admission that such prior art is widely known or forms part of the common general knowledge in the field.

### SUMMARY OF THE INVENTION

The objective of the present invention was, hence, to enrich or improve the state of the art and in particular to provide the art with a method to efficiently degrade polyurethane in packaging material, for example a polyurethane layer in a multi-layer packaging that does not require prior separation of layers, does not require harsh chemicals and/or harsh conditions, and offers economic and environmental advantages, or to at least provide a useful alternative to solutions available in the art.

The inventors were surprised to see that the objective of the present invention could be achieved by the subject matter of the independent claim. The dependent claims further develop the idea of the present invention.

Accordingly, the present invention provides a method of degrading polyurethane (PU) comprising the step of subjecting the PU to at least one cutinase.

As used in this specification, the words “comprises”, “comprising”, and similar words, are not to be interpreted in an exclusive or exhaustive sense. In other words, they are intended to mean “including, but not limited to”.

The present inventors have shown that cutinases can efficiently be used to degrade PU in packaging material. The inventors have obtained particular promising results with the cutinases BC-CUT-013 and Thc_Cut1. Remarkably, cutinases could be used to selectively degrade PU-containing layers in multilayer packaging. For example in the case of PE based multilayer packaging structure that comprises a PU-based layer, it was possible by using cutinases to selectively degrade the PU-based layer, so that the PU monomers could be recovered, and the PE-based backbone of the multilayer packaging structure could be liberated and subjected to PE recycling. The clean state of the resulting PE allowed that the recycled PE could be recycled for high-value applications. In particular, the inventors have realized that the cutinases BC-CUT-013 and Thc_Cut1 have a substantial effect on degradation of polyurethanes that are deprived of urea linkages. With this, the inventors surprisingly achieved degradation of non-biodegradable PU with very low enzyme loading (polymer-to-enzyme ratio) that are lower than reported elsewhere. BC-CUT-013 is originated from Aquabacterium fontiphilum.

### BRIEF DESCRIPTION OF THE DRAWINGS

Additional features and advantages of the present invention are described in, and will be apparent from, the description of the presently preferred embodiments which are set out below with reference to the drawings in which:

FIG. 1 is a diagram illustrating single enzyme degradation in % of commercial solvent-based adhesive Adcote 102A (white) and Adcote 545-75 (grey) as well as coating Adcote 17-3 (black) after 17 days of reaction with an enzyme loading of 5.6-7 μg/mg polymer. All reactions were carried out in 1.5 ml 0.1 M PBS buffer set to pH 7 at 250 rpm and 37° C. For a negative control, only buffer was added to the vials; the positive control contained 1M NaOH instead of enzyme. All reactions were performed in duplicates.

FIGS. 2A, 2B and 2C are diagrams illustrating enzymatic degradation profile of BC-CUT-013 (diamond symbol, ⋄) for the three different PU materials Adcote 102A (A), Adcote 545-75 (B) and Adcote 17-3 (C) as degree of degradation based on FDL release. All reactions were carried out at 37° C. using 20-26.6 mg PU in 1.5 ml glass tubes containing 0.1 M PBS buffer set to pH 7 and shaken at 250 rpm. For the negative control, only buffer was added to vials (●); the positive control consisted of 1M NaOH (▪ dashed). Each symbol represents the average of reactions performed in duplicate. The enzyme loaded of crude powder was 6-7.5 μg protein/mg polymer.

FIGS. 3A and 3B are diagrams that show degradation efficiency of BC-CUT-013 cutinase depending on reaction pH. Degradation percentage (%) expressed as release of degradation product (FDL) after 14 days using 20-25 mg commercial polyurethane material Adcote 545-75 (A) and Adcote 17-3 (B) at pH 6.5 (white bars), 7 (light grey bars), 7.5 (dark grey bars) and 8 (black bars). A negative control was performed each time with polyurethane solely in 0.1 M PBS buffer for each respective pH. The reactions were carried out in 1.5 ml at 37° C. and enzyme loadings of 5.6 μg/mg polymer. Each bar represents the average percentage of degradation product (FDL) released duplicate reactions. For a negative control, only buffer was added to vials; the positive control contained of 1M NaOH

FIGS. 4A and 4B are diagrams that illustrate kinetics of polyurethane degradation for BC-CUT-013 cutinase depending on pH. Degradation percentage (%) is expressed as release of degradation product (FDL) after 14 days of enzymatic hydrolysis of 20-25 mg commercial polyurethane material Adcote 545-75 (A) and Adcote 17-3 (B) at pH 6.5 (diamond symbol, ⋄), 7 (circle, ◯), 7.5 (triangle, Δ) and 8 (square, □). A negative control was performed each time with polyurethane solely in 0.1 M PBS buffer for each respective pH. The reactions were carried out in 1.5 ml at 37° C. and enzyme loadings of 5.6-7 μg/mg polymer. Each symbol represents the average percentage of degradation product released for duplicate reactions. For a negative control, only buffer was added to vials (filled circle, ●); the positive control consisted of 1M NaOH (▪ dashed).

FIGS. 5A and 5B are diagrams depicting enzymatic degradation using enzyme combination of BC-CUT-013 and Thc_Cut1. The reactions were carried out on 0.79 mg Adcote 17-3 in 0.2 ml at 37° C. and enzyme loadings of 25.6 μg protein/mg for the single enzymes and each enzyme in combination (in total 51.2 μg/mg polymer). (A) Total polymer release profile of enzymatic hydrolysis with BC-CUT-013 (diamond, ⋄) and BC-CUT-013+Thf_Cut1 (filled triangle, Δ). For a negative control, only buffer was added to vials (filled circle, ●); the positive control consisted of 1M NaOH (▪ dashed). (B) Depicts the difference of the combination (white bars) and single enzyme activities (grey and black bars, respectively) after a reaction time of 24 h. A negative control was performed each time with polyurethane solely in 0.1 M PBS buffer at pH 7. Each bar represents the average percentage of degradation product released for duplicate reactions.

FIG. 6 is a diagram that illustrates degradation product profiles of total products (mM) after enzymatic hydrolysis of post-consumer water bottles with 30% recycled PET determined by HPLC. The reactions were carried out at 37° C. and pH 7 for 7 days using 20-25 mg substrate grounded to 0.2-0.5 mm. Enzyme loading of 5.6-7 μg protein/mg polymer was used for the enzymes: Thf_Cut (diamond, ⋄), Thc_Cut2 (triangle, Δ), Thc_Cut1 (circle, ◯) and BC-CUT-013 (square, □). A negative control was performed each time with polyurethane solely in 0.1 M PBS buffer at pH 7. Each bar represents the average percentage of degradation product released for duplicate reactions.

FIGS. 7A and 7B are diagrams showing enzymatic hydrolysis of post-consumer water bottles with 30% recycled PET. The reactions were carried out using an enzyme loading of 5.6-7 μg protein/mg polymer at 37° C. and pH 7 for 2 days (A) and 7 days (B) with 20-25 mg substrate grounded to 0.2-0.5 mm. The bars indicate the concentration of the hydrolysis products TPA (white bars), MHET (grey bars) and BHET (black bars) in the reaction mixture determined by HPLC. Each bar represents the average of reactions performed in duplicate.

FIG. 8 illustrates identification of enzymatic degradation products for Thf_cut1 on Adcote 102A by LC-HRMS analysis of enzymatic reaction solution after 17 days (37° C. and pH 7). All TIC plots (A-C) were recorded in positive mode with m/z ratios ranging from [80.0000-1200.0000]. (A) TIC plot of degradation products stemming from enzymatic reaction of Thf_cut1 5.64-7 μg protein/mg polymer with Adcote 102A (B) TIC plot product release without enzymatic treatment (negative control, only 0.1 M PBS buffer) is depicted after 17 days of reaction. (C) TIC plot of the 1M NaOH positive control after 17 days of reaction. (D) Representation of structures I-III of identified compounds, mainly sebacic acid based fragments.

FIG. 9 depicts identification of enzymatic degradation products for BC-CUT-0013 on Adcote 545-75 by LC-HRMS analysis after 17 days of reaction at 37° C. and pH 7. All TIC plots (A-C) were recorded in positive mode with m/z ratios ranging from [80.0000-1200.0000]. (A) TIC plot of degradation products stemming from enzymatic reaction of BC-CUT-013 (enzyme loading of 5.6-7 μg protein/mg polymer) with Adcote 545-75 (B) TIC plot product release without enzymatic treatment (negative control, only 0.1 M PBS buffer) is depicted after 17 days of reaction. (C) TIC plot of the 1M NaOH positive control after 17 days of reaction. (D) Structures I-VII of all identified compounds, all three monomers (DEG, adipic acid, phthalic acid) and four phthalic acid-DEG based dimers/fragments (IV-VII).

FIG. 10 illustrates identification of enzymatic degradation products for BC-CUT-0013 on Adcote 17-3 after 17 days of reaction at 37° C. and pH 7. All TIC plots (A-C) were recorded in positive mode with m/z ratios ranging from [80.0000-1200.0000]. (A) TIC plot of degradation products stemming from enzymatic reaction of BC-CUT-013 on Adcote 17-3 (enzymatic load 5.6-7 μg protein/mg polymer). (B) TIC plot product release without enzymatic treatment (negative control I, only 0.1 M PBS buffer) is depicted after 17 days of reaction. (C) TIC plot of the 1M NaOH positive control after 17 days of reaction. (D) Structures I-VII of all identified compounds, all three monomers (DEG, adipic acid, phthalic acid) and four phthalic acid-DEG based dimers/fragments (IV-VII).

### DETAILED DESCRIPTION OF THE INVENTION

Consequently, the present invention relates in part to a method of degrading polyurethane (PU) in packaging material comprising the step of subjecting the packaging material comprising the PU to at least one cutinase.

The PU may be provided as pure material or as a material comprising PU.

The inventors have obtained, for example, very good results, when the material comprising PU was a polyester-containing polyurethane-based polymer. For example, the inventors have obtained excellent results with coatings and adhesives that are polyurethane-based with aliphatic and aromatic polyester segments.

In accordance with the present invention the PU is degraded by at least one cutinase. The term “degradation” comprises the fragmentation of the polymer matrix though de-polymerization, which refers to the process of converting a polymer into smaller polymer chains, oligomers and eventually monomers. The term “degradation” more generally describes that the polymer chain is cleaved by at least one of the enzymes, resulting in shorter polymer chains with or without the release of monomers. Such polymer fragmentation can for example be achieved through the activity of endo-acting enzymes or through the incomplete activity of exo-acting enzymes. In one embodiment of the present invention the method of the present invention may be a method of de-polymerizing PU, for example at least one PU-based layer in a packaging.

Cutinases catalyze the hydrolytic reaction of cutine and water to yield cutine monomers. Cutinases belongs to the family of serine esterases, typically containing the Ser-His-Asp triad of serine hydrolases.

The at least one cutinase may be a cutinase from a fungal or microbial source. Using enzymes from a fungal or a microbial source have the advantage that they can be naturally produced, and—in particular, if the enzymes are enzymes that are secreted by the fungus or the microorganism—the fungus or the microorganism itself can be used to degrade the at least one polymer layer in a packaging material.

The at least one cutinase may be a cutinase from Thermobifida fusca or Thermobifida cellulosilytica, or Thermobifida alba.

Thermobifida organisms are a thermophilic organism occurring in soil that is a major degrader of plant cell walls in heated organic materials such as compost heaps, rotting hay, manure piles or mushroom growth medium. Its extracellular enzymes have been studied because of their thermostability, broad pH range and high activity.

The inventors have obtained particularly promising results, when the at least one cutinase was selected from the group consisting of cutinase-like esterase BC-CUT-013, Thf_Cut1, or Thc_Cut2. These cutinases produced even better results than other cutinases.

Thc_Cut2 (T. cellulosilytica), Thf_Cut1 (T. fusca) as well as the metagenomic cutinase BC-CUT-013 were purchased from Biocatalyst Ltd. UK and were recombinantly produced in E. coli.

The enzymes may be used in pure form. However, the inventors were surprised to see that the enzymes could also be used as crude extracts, for example, as crude extract from a fungal and/or microbial source. Using a crude extract has the advantage that an expensive purification of the enzymes is not necessary. Consequently, in accordance with the present invention the at least one cutinase may be used as a crude extract. Advantageously, the at least one cutinase may be used as a water soluble, crude extract.

The amount of enzyme used is not critical for the success of the degradation step in the method of the present invention. It is, however, important for the speed of the degradation. The inventors have obtained good results when the degradation was carried out with an enzyme concentration of at least about 0.65 μg protein/mg polymer, at least about 6.5 mg protein/mg polymer, or at least about 50 μg protein/mg polymer.

In particular if the cutinase used in the framework of the present invention is obtainable from a thermophilic organism, the cutinase will also exhibit a certain thermo-stability. Accordingly, the degradation can be carried out at elevated temperatures, for example at a temperature in the range of 30-40° C., 35-45° C. or 40-50° C. The degradation at elevated temperatures will proceed significantly faster. The expected increase in reaction speed can be estimated in accordance with Arrhenius law.

However, elevating the reaction temperature will cause costs, for example for the increase in energy usage. Hence, it may be preferred if the degradation is carried out at ambient temperature. This is, in particular, the case if the required reaction time is not critical. Ambient temperature may differ depending, for example, on geographic location and on the season. Ambient temperature may mean for example a temperature in the range of about 0-30° C., for example about 5-25° C.

Accordingly, for example, in the framework of the resent invention, the PU may be subjected to the at least one cutinase at a temperature in the range of 20-50° C., for example 30-40° C. The inventors have obtained very good results at a temperature of about 37° C.

The inventors have further tested the reaction at different pH values. It was found that the method of the present invention was most effective, if the degradation was carried out at neutral to slightly alkaline conditions. Good results were obtained at a pH in the range of 6-9. For example, the PU may be subjected to the at least one cutinase at a pH in the range of about 6-9, for example in the range of about 6.5-8.

Accordingly, it may be preferred if the degradation is carried out at pH in the range of about 7-9, preferably in the range of about 7.5-8.5, for example at a pH of about 8.2.

The inventors have obtained good results when the PU was subjected to the at least one cutinase for at least 3 days, for at least 10 days, or for at least 20 days.

With the method of the present invention a partial or even a complete degradation of the PU appears possible. The inventors conclude this from a corresponding release of reporter molecules. For example, it appears possible with the method of the present invention to degrade the PU by at least 10 weight-%, at least 15 weight-%, at least 20 weight-%, at least 25 weight-%, at least 30 weight-%, at least 35 weight-%, at least 45 weight-%, at least 50 weight-%, or at least 55 weight-%. This degradation resulted in part in the generation of monomers or monomer mixtures. Accordingly, in the method of the present invention the degradation of the at least one polymeric layer results in the generation of at least 10 weight-%, at least 15 weight-%, at least 20 weight-%, at least 25 weight-%, at least 30 weight-%, at least 35 weight-%, at least 45 weight-%, at least 50 weight-%, or at least 55 weight-% of the monomers or monomer mixtures of the degraded polymer.

The method of the present invention is—in particular—well suited for application in packaging recycling. Accordingly, in the framework of the present invention, the PU may be present in a packaging, for example in food packaging or pet food packaging. For the purpose of the present invention, the term “food” shall be understood in accordance with Codex Alimentarius as any substance, whether processed, semi-processed or raw, which is intended for human consumption, and includes drink, chewing gum and any substance which has been used in the manufacture, preparation or treatment of “food” but does not include cosmetics or tobacco or substances used only as drugs.

Multilayer packaging structures are frequently used in the industry today, for example in the food industry. Here, multi-layered packaging is often used to provide certain barrier properties, strength and storage stability to food items. Such a multi-layered packaging material may be produced by lamination, or coextrusion, for example. Further, techniques based on nanotechnology, UV-treatments and plasma treatments are used to improve the performance of multi-layer packaging. Compr Rev Food Sci Food Saf. 2020; 19:1156-1186 reviews recent advances in multilayer packaging for food applications.

If the packaging comprises a multi-layer packaging material, the multi-layer packaging material may comprise at least two polymeric layers.

The polymeric layers may comprise a PU-based layer and at least one layer selected from the group consisting of a further PU-based layer, a polyethylene terephthalate (PET)-based layer, a polyethylene (PE)-based layer, or a combination thereof. The PU-based layer may be a PU-based adhesive or a PU-based coating.

A layer shall be considered PU, PE or PET based, if it contains at least about 50 weight-%, at least about 60 weight-%, at least about 70 weight-%, at least about 80 weight-%, at least about 90 weight-%, at least about 95 weight-%, or at least about 99 weight-% of PU, PE or PET, respectively.

PU layers are frequently used in food packaging as adhesive or coating, for example. PU layers are typically applied in flexible films requiring high elongation, inherently strong, flexible, and free of plasticizers, that do not become brittle with time.

PET layers are also frequently used in food packaging. They are transparent, have a very good dimensional stability and tensile strength and are stable over wide temperature ranges. PET layers do not absorb water, are UV-resistant and provide a good gas barrier. Furthermore, it is easy to print on PET in high quality. The gas barrier properties of PET films are, however, only moderate. Today's mechanical recycling technologies for PET yield lowered recyclate quality and depending on the feedstock (e.g., for mixed PET waste) limited food grade application.

Polyethylene (PE) is a plastic polymer belonging to the polyolefin family that is relatively easy to recycle mechanically, nowadays. As a thermoplastics with carbon-carbon polymer chain, PE becomes liquid at their melting point and do not start to degrade under elevated temperatures as compared to thermoplastics with hydrolysable bonds, such as PET. Hence, such polyolefins thermoplastics can be heated to their melting point, cooled, and reheated again without significant degradation. Upon liquification of PE due to heat, PEs can be extruded or injection molded and—consequently—recycled and used for a new purpose. However, it is problematic to recycle PEs if—e.g., in a multi-layer packaging material—a PE layer is combined with other plastic layers.

One advantage of the method described in the present invention is that it can be used to delaminate selectively PU layers from a PE layer. Consequently, the method of the present invention may be used for the selective delamination of at least one PU-based layer in a multilayer packaging.

The inventors could show that the enzyme(s) used in the framework of the present invention could degrade PU-based layers. For example, the inventors have shown that commercially available polyurethanes could be degraded with the cutinases used in the framework of the present invention.

In the method of the present invention, the PU may be present in a packaging comprising a multilayer packaging structure, wherein the multilayer packaging structure comprises a base layer that can be recycled, for example a PE-based layer, and at least one PU-based layer, wherein the method is used to recycle the multilayer packaging structure by degrading the at least one PU-based layer and by subjecting the base layer to a recycling stream. The resulting PU monomers can be collected and reused as well.

Many multilayer packaging structures comprise a PE-based layer, a PET-based layer and a PU-based layer. The inventors have shown that cutinases can be used to degrade PU-based layers. The use of cutinases to biodegrade PET is known, for example, from Nature Scientific Reports (2019) 9:16038. Consequently, in one embodiment the present invention relates to a method of degrading multilayer packaging structures comprising at least one PU-based layer and at least one PET based layer comprising the step of subjecting the multilayer packaging structure to at least one cutinase.

In a further embodiment of the method of the present invention, the packaging comprises a multilayer packaging structure comprising at least three polymeric layers, wherein the polymeric layers comprises at least one PU-based layer, at least one PET based layer and at least one PE-based layer wherein the method comprises the step of subjecting the multilayer packaging structure to at least one cutinase, and subjecting the PE-based layer to further recycling. The generated building blocks of the PU-based layer and/or the PET-based layer may be collected for reuse.

In scope of the presented invention, the inventors also propose its application for multilayer packaging that are comprised of more than three polymeric layers. For example, polyvinyl alcohols (PVHOs), such as EVOH and BVOH used for oxygen barrier, are typically found in addition to PU-, PET- and PE-layers and would be released from the multilayer besides PE when subjected to at least one cutinase as described in this invention.

The inventors further propose that the degradation speed and/or completeness can be significantly increased, if the surface to volume ratio of the packaging, for example the multilayer packaging structure is increased. For example, the packaging may be mechanically treated to reduce the particle size to particles with an average diameter of less than about 5 mm, less than about 1 mm, or less than about 0.5 mm diameter before subjecting the packaging to the enzyme. Typically, the mechanical treatment may be shredding, for example. Hence, the method of the present invention may further comprise the step of reducing the particle size of the PU and/or the PU containing material, for example the PU containing packaging, before or during subjecting the PU and/or the PU containing material to at least one cutinase. The particle size may be reduced by a mechanical treatment to particles with an average diameter of less than about 5 mm, less than about 1 mm, or less than about 0.5 mm diameter.

One advantage of the method of the present invention is that it can be carried out under controlled conditions, for example in a closed vessel, such as a bioreactor, for example. The relatively gently conditions of the degradation process do not require bioreactors that can withstand extreme conditions, which in turn contributes to the safety and cost effectiveness of the method of the present invention. Using a closed vessel in turn has the advantage that reaction and process parameters, such as temperature and agitation, for example, can be precisely controlled.

Those skilled in the art will understand that they can freely combine all features of the present invention disclosed herein. In particular, features described for the method of the present invention may be combined. Further, features described for different embodiments of the present invention may be combined.

Although the invention has been described by way of example, it should be appreciated that variations and modifications may be made without departing from the scope of the invention as defined in the claims.

Furthermore, where known equivalents exist to specific features, such equivalents are incorporated as if specifically referred in this specification. Further advantages and features of the present invention are apparent from the figures and non-limiting examples.

### EXAMPLES

### Example 1: Cutinases Degrading Commercial Polyurethane-Based Adhesives and Coating

Material and Methods

Materials and Chemicals

The polyurethane materials Adcote 102A (36% w/w), Adcote 545-75 (75% w/w), Adcote 17-3 (75% w/w) and co-reactant F (75% w/w) were thankfully provided by Dow Chemicals. Glycerol, K2HPO4, KH2PO4, fluorescein, fluorescein dilaurate, sodium hydroxide (NaOH) and ethyl acetate were all purchased from Sigma.

Based on analysis of degradation products by Liquid chromatography high resolution mass spectrometry (LC-HRMS) following monomer contents could be confirmed (see Table 1). All materials contain phthalic acid as well as diethylene glycol. Adcote 102 A and Acote 17-3 also contain both sebacic acid as diacid component, whereas Adcote 545-75 contains adipic acid. For the coating neo-pentyl-di-propanol could be detected. Co-reactant F was described in patents to contain isocyanate terminated polyol based branched pre-polymers. The isocyanate component was found to be toluene di-isocyanate (Wu et al, 2019, US20190284456A1).

TABLE 1
List of the three materials tested (Adcote 102A, Adcote 545-75 and Adcote 17-3) their preparation and
identified components by LC-MS, which were typically identified as enzymatic
degradation products in this invention.

| Name | Adcote 102A | Adcote 545-75 | Adcote 17-3 | Co-Reactant F |
|---|---|---|---|---|
| Description | Polyester | Polyester | solvent-based | Cross-linked, |
| | component of | component of | coating | isocyanate |
| | two component | two component | | containing |
| | based adhesive | based adhesive | | component |
| Preparation | cured with co- | Cured with co- | Already cured | Cured with |
| | reactant F | reactant F | | polyester |
| | | | | component |
| Identified Components | 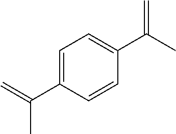 | 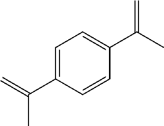 | 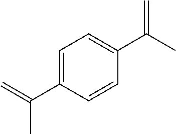 | 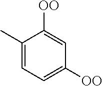 |
| | 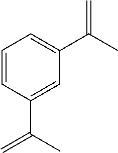 | 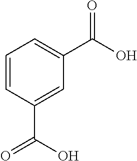 [SMILES: O=C(O)c1cccc(C(=O)O)c1] | 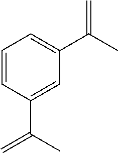 | 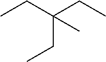 |
| | 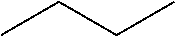 | 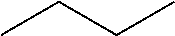 | 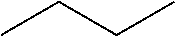 | 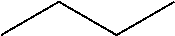 |
| | 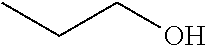 | 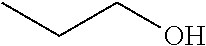 | 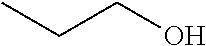 | 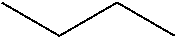 |
| | 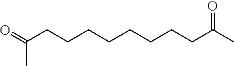 | 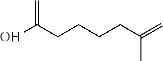 | 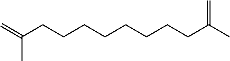 | |
| | | | 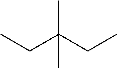 | |

Thf_Cut1 (T. fusca), Thc_Cut2 (T. cellulosilytica) and ThcCut1 (T. cellulosilytica) as well as the metagenomic cutinase BC-CUT-013 were purchased from Biocatalyst Ltd. UK. The enzyme BC-CUT-013 was identified via a metagenomic search against the query amino acid sequences from Thermobifida fusca CUT1.

TABLE 2
List of enzymes investigated, enzyme family, abbreviation, organism
of origin, production organism, quality and supplier.

| | | | | Production | | Supplier/ |
|---|---|---|---|---|---|---|
| Name | Abbreviation | Family | Organism | Organism | Quality | Producer |
| T. fusca cutinase | Thf_Cut | Cutinase | T. Fusca | Recombinant | Crude | Biocatalysts |
| T. cellulosilytica | Thc_Cut2 | Cutinase | T .cellulosilytica | E. coli | soluble | Ltd. UK |
| cutinase 2 | | | | | extract | |
| T. cellulosilytica 1 | Thc_Cut1 | Cutinase | T. cellulosilytica | | | |
| Biocatalyst | BC-CUT-013 | Cutinase | Metagenomic | | | |
| Cutinase 013 | | | | | | |

All enzymes were diluted to stock solutions of 1 mg/ml protein in 40% (w/v) glycerol for easier handling during experiments.

The degree of adhesive and coating degradation by enzymes was measured via the following methods: fluorescent release assay for indirect estimation of polymer degradation and LC-MS identification of specific degradation products proofing hydrolysis into polymer building blocks (oligomers and monomers).

Upon receiving of the polymer materials, 2.5× (polyester component) and 5× (co-reactant) stock solutions (w/w) were prepared by diluting the polymer in ethyl acetate.

For the adhesives Adcote 102A and Adcote 545-75 the co-reactant had to be mixed with the polymer in ratios of 4.5:100 (w/w) and 11.5:100 (w/w) respectively.

Preparation of Polyurethane Coated Glass Vials

The indirect fluorescent assay, established by Zumstein and colleagues (Zumstein, M. T., et al. (2017) Environmental Science & Technology 51(13): 7476-7485) is based on the assumption that the release of a homogeneous embedment reporter molecule (fluorescein dilaurate, FDL) in the target polymer matrix (adhesive or coating) is directly correlated to the degree of degradation of same polymer material. Only upon material degradation, FDL is released out of the polymer matrix and can then be hydrolyzed by an esterase-active enzyme into laureate and fluorescein, of which the latter molecule can be quantified fluorometrically (521/494 nm). One percent (%) polymer degradation is defined as one % release of originally embedded reporter molecule, in this case corresponding to 0.1 wt % incorporated FDL which was the optimum amount to reach a high detection limit while minimizing the effect on the polymer matrix and enzymes.

The stock solutions were used to prepare the casting solutions in ethyl acetate containing 12.6% (w/w) polymer and 0.0126% (w/w) FDL. This corresponds to a FDL:polymer ratio of 1:1000.

For 26.6 mg polymer, 200 μl of the casting solution were transferred into 2 ml HPLC vials (Agilent), vortexed for 2 s and then dried in a falcon rotator (Rotary-mixer 34526, Snijders) for 5-6 h. This was done to ensure an evenly spread coating on the inner side of the vial. As both Adcote 102A and 545-75 constitute glues this ensured a clean even surface for the reactions.

The vials were then dried at 50° C. for 1 h before leaving them to cure for 1 week at room temperature.

Single Enzyme Activity Screening of Four Enzymes Against Adcote 102A, 545-75 and 17-3

The reaction was carried out in 1.5 ml 0.1 potassium-saline phosphate-Buffer (pH 7) with an enzyme load of 5.6-7 μg protein/mg polymer. This was done to ensure pH stability as acids are formed upon hydrolysis that may affect the enzyme negatively. The buffer was prepared by mixing K₂HPO₄ and KH₂PO₄ according to the Henderson-Hasselbalch equation.

As a positive control reaction for the assay, the FDL-loaded polymer sample was exposed to a 1M sodium hydroxide (NaOH) solution as stability of long-chain fluorescein diesters and greatly decreases above pH 8.5 (Guilbault, G. G., & Kramer, D. N. (1966), 14(1), 28-40). In addition, ester bonds as in polyesters as well as urethanes bonds present in polyester-polyurethanes can be hydrolysed at elevated pH as reported in a study by Matuszak and colleagues (Matuszak, M. L., Frisch, K. C., & Reegen, S. L. (1973), Journal of Polymer Science: Polymer Chemistry Edition, 11(7), 1683-1690). Thus, a basic solution of 1M NaOH is used as positive control for the indirect FDL assay.

As a negative control, FDL-loaded polymer sample were exposed to the respective buffer solution without enzyme or NaOH. Leakage of FDL was determined negligible.

All vials were incubated at 37° C. at 250 rpm. Samples of 50 μl were taken after 0, 1, 2, 3, 3.5, 14, 15, 16 and 17 days, mixed with 150 μl 4M NaOH and measured at 494/521 nm in a plate reader. This was done to ensure full hydrolysis of all free released FDL. A fluorescein calibration curve of 3.125-5 μM was used to calculate the FDL release.

Additional samples of 50 μl for LC-HRMS were taken at 0, and 28 days and added to 205 microliter to 25 mM HCl in the HPLC mobile phase (0.1% formic acid in 30% MeOH). After 14 days the reaction solution was replaced with fresh enzyme solution, finally after 28 days the reaction was stopped and all vials stored at −20° C.

LC-HRMS

The analysis of polyurethane based degradation products has been performed by LC-ESI-HRMS. Samples of 50 μl have been collected before starting the reaction and after 14 days of reaction. For analysis, samples were defrosted and centrifuged for 10 min at 12000 rpm to precipitate any solid particles. The supernatant was injected directly without any further pre-treatment.

Samples were separated by reversed phase chromatography on an ACQUITY-biocompatible Transcent HPLC system (Thermo Fisher Scientific) equipped with a Waters Acquity UPLC BEH C8 column (ID 2.1×100 mm, 1.7 μm) and with a Waters Acquity UPLC BEH C8 guard column (2.1×50 mm, 1.7 μm). A gradient elution system consisted of an aqueous mobile phase (A) (0.5 mM Ammonium formate and 0.1% formic acid) and mobile phase (B) (methanol containing 0.1 formic acid and 0.5 mM ammonium formate) with a flow rate of 0.4 ml/min. The gradient was initiated at 5% B increased to 45% B at 0.25 min, to 100% B by 1 min and kept at 100% B for 15 min. The column oven temperature was set to 40° C. The HPLC flow after the analytical column is splitted (ratio˜1:9) for mass and DAD-CAD detection respectively. A reverse gradient is applied for CAD detection (Corona Veo RS Charged Aerosol Detector, Thermo Fisher Scientific) to compensate the drift of gradient composition.

The HPLC eluent was directly electrosprayed from the column end at an applied positive spray voltage of 3.5 kV, using a sheath gas flow rate of 15 L/h and an Aux gas flow rate of 5 L/h. The capillary temperature was set to 250° C. the aux gas heater temperature to 100° C. The chromatographic system was coupled to a Q-Exactive Classic, (Thermo Fisher Scientific). The full MS survey scans were acquired at 35000 resolution power over the mass range of 80-1200 m/z. Peaks were analyzed in positive mode in a mass range m/z [80.0000-1200.0000] using Xcalibur software (version 4.2.47, Thermo Scientific).

Results and Discussion

For the recycling of laminates and polyurethane coated packaging, the selective degradation of the polyurethane layer is the key to separate layers and enabling their subsequent individual recycling. Furthermore, most of enzymatic degradation studies of polyurethanes studies have been carried out on custom-made PU polymers and not on commercial industrially relevant PU polymers and formulations. This may be due to the much more complex and diverse chemical composition, especially in protected commercial formulations that complicates the analysis of enzymatic degradation process.

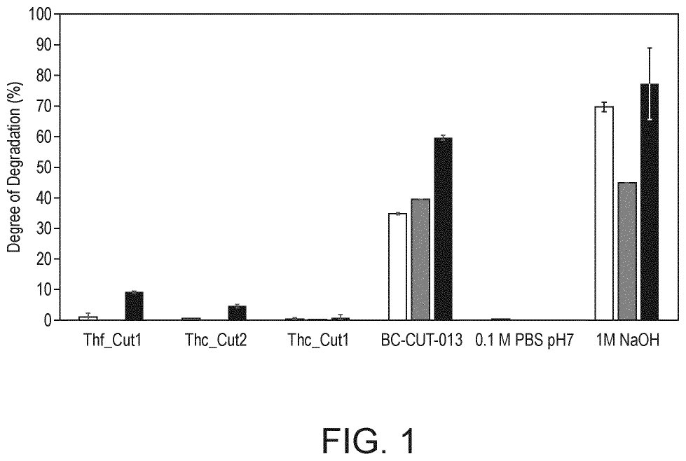

The inventors screened 4 enzymes towards their activity on the commercial PU materials including the already mentioned Thc_Cut, Thf_cut1, Thf_cut2, and BC-CUT-013 (see FIG. 1).

As can be seen in FIG. 1, after 17 days of reaction at controlled 37° C., all polyurethane (PU) materials were effectively degraded by at least one or more of the tested enzymes using an enzyme loading of 5.6-7 μg protein per mg polymer (μg/mg). The results also confirm that 1M NaOH can be used as positive control reaction for this assay. The inventors point out that NaOH was purely used as positive controls for analytical purposes and not as alternative treatment option to enzymatic delamination. NaOH treatment as opposed to enzymatic degradation is non-selective and generates enantiomer mixtures of products, imposes harsh process conditions and process setup as well as safety, and requires additional downstream processing operations (e.g., neutralization and removal) that increases overall process costs and makes it thus a non-viable and non-suitable method.

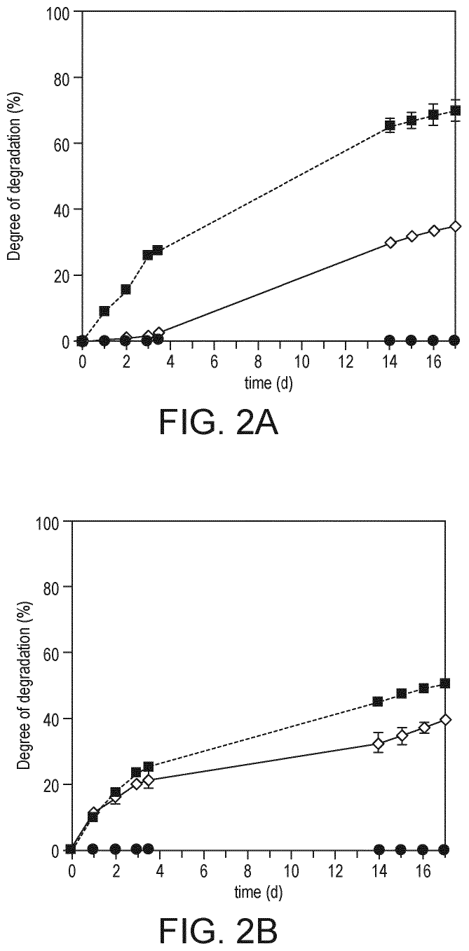

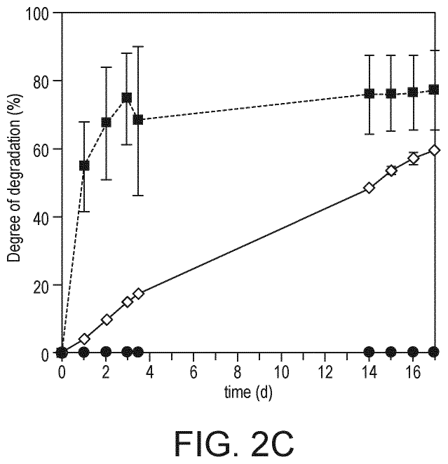

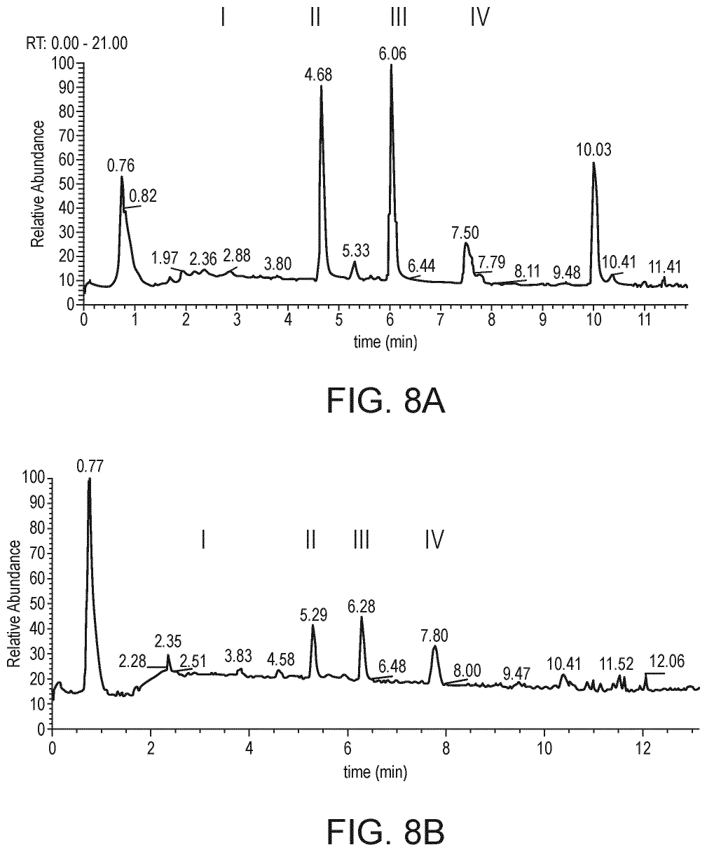

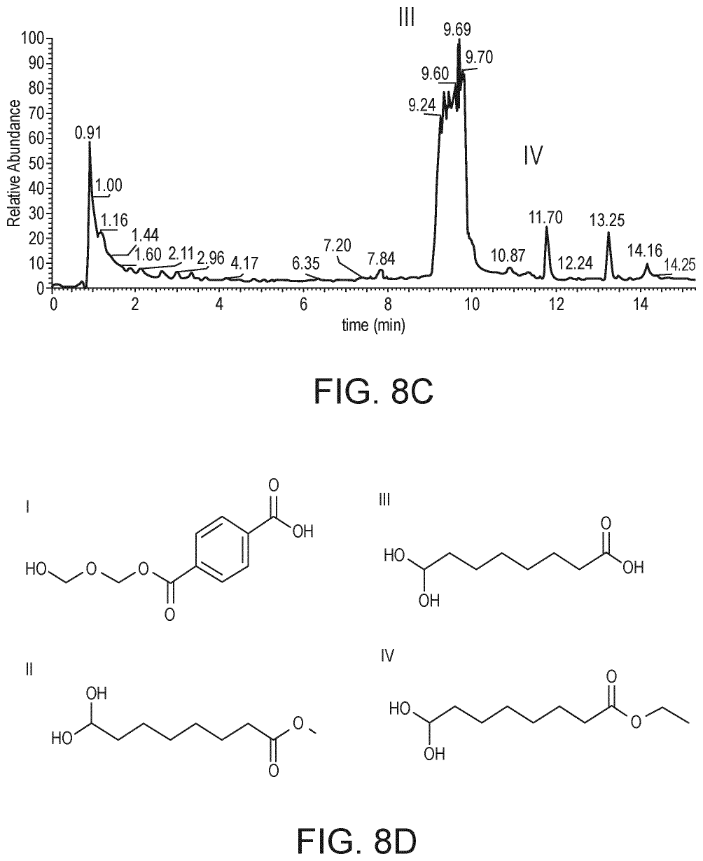

As evident from FIGS. 1 and 2, the metagenome-identified cutinase BC-CUT-013 demonstrated highest degradation activity with 60%, 39% and 34% for the commercial PU coating Adcote 17-3, the adipic acid containing Adcote 545-75 and the sebacic acid containing adhesive Adcote 102A, respectively (based on the matrix release of FDL and specific degradation products identified). LC-HRMS analysis confirmed PU degradation by identifying the individual monomers and degradation products (see FIGS. 8-10) The positive control with 1M NaOH reached 70%. Clearly, BC-CUT-013 shows highest activity on the three different PU materials, however, Thf_Cut1 and Thc_Cu2 also showed some degradation activity (ca. 10% and 5%) on the coating 17-3, which are in a similar range reported previously (Schmidt, J., et al. (2017). Polymers 9(2): 65) for the commercial thermoplastic polyester PU Elastollan B85A-10 and C85A-10, yet, these PU materials are not typically used in packaging applications.

FIG. 2 shows the kinetics of the enzymatic PU degradation using the cutinase BC-CUT-013 that demonstrated highest degradation efficiency and continuous increase in product release for all three PU materials (FIGS. 2A-C). The cutinase BC-CUT-013 was the most active enzyme on the adipic acid containing adhesive Adcote 545-75 (see FIG. 2B) and on the coating Adcote 17-3 (see FIG. 2C) with 39 and 60% polymer degradation, respectively. Of all materials, Adcote 102A was hydrolyzed the slowest by BC-CUT-013, but still reaching 34% polymer degradation after 17 days of reaction see FIG. 1 and FIG. 2A).

For all materials the reaction rates were highest for the first three days of incubation before the degradation rate decreased slightly. Replacing the BC-CUT-013 enzyme solution after 14 days, faster degradation rates could be obtained for Adcote 545-75 (FIG. 2B). The control reaction (negative control) showed no significant degradation (see. FIGS. 1 and 2) proofing the efficiency of enzymatic-catalyzed degradation of the commercial PU materials.

In this invention, the inventors demonstrate the efficient cutinase-catalyzed degradation of commercial, solvent-based polyurethane adhesives (Adcote 102A, Adcote 545) and the coating Adcote 17-3 from DOW Chemicals at surprisingly mild reaction conditions of 37° C. at pH 7 and very low crude enzyme loadings (5.6-7 μg protein per mg PU material).

Identification of Degradation Products by LC-HRMS

The degradation products of the commercial adhesives and coating shown in FIGS. 8-10 were identified via LC-HRMS proving the successful hydrolysis of the three different polyurethane polymers through the active cutinase enzymes (FIGS. 1 and 2) as well as positive control. The identified degradation products mainly differ depending on the polyurethane polymer and thus indicate a different composition. These results confirm that the FDL represents a good indirect method to test enzymatic degradation of PU polymer matrixes.

FIG. 8 represents the chemical identification of water-soluble degradation products via LC-HRMS for the reaction with Thf_cut on Adcote 102A demonstrating the ability of this cutinase to cleave and release degradation products consisting mainly of sebacic acid based fragments (see FIG. 8, compounds 1-III).

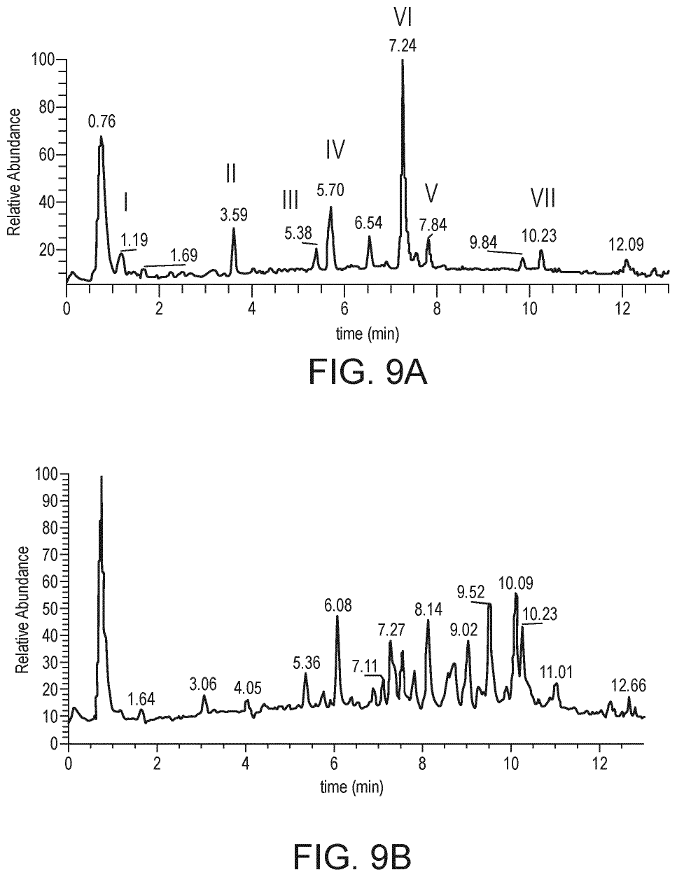

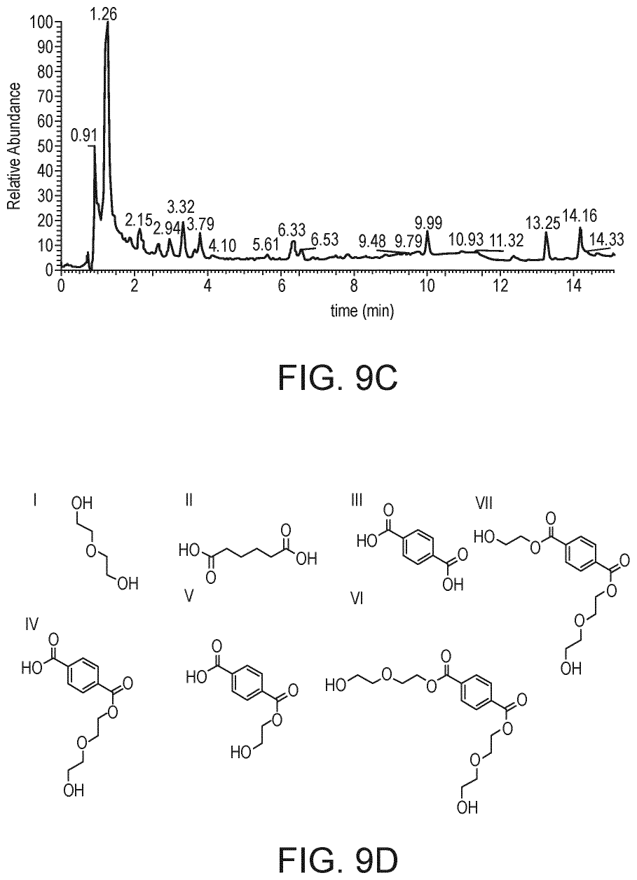

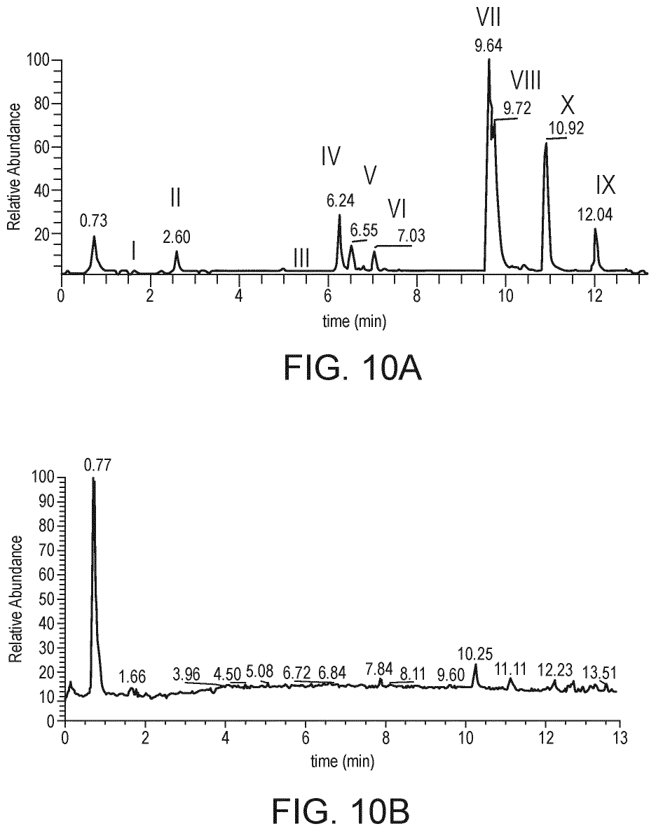

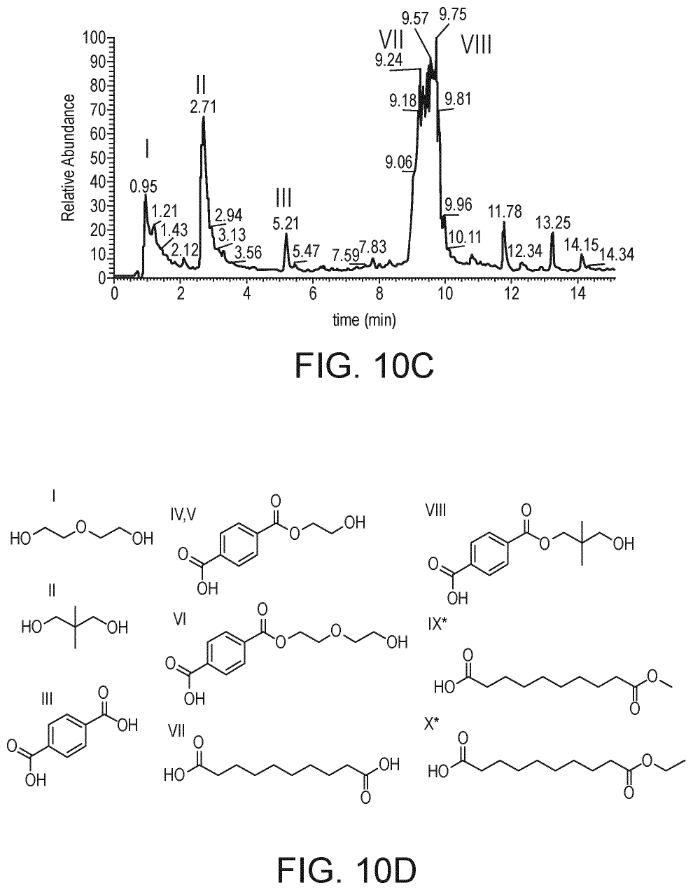

FIGS. 9 and 10 show the specific degradation products for both Adcote 545-75 and Adcote 17-3 were detected when using BC-CUT-013 as biocatalyst.

For Adcote 545-75 (FIG. 9) seven degradation products were detected, including the monomers diethylene glycol (DEG), adipic acid and phthalic acid as well as several phthalic acid-DEG based fragments. Of all three materials, Adcote 17-3 showed the most variety of degradation products (see FIG. 10). Apart from the monomers (sebacic acid, phthalic acid, DEG and neopentyl glycol) there were three larger sebacic acid-containing fragments as well as three phthalic acid based dimers.

Only negligible amounts of degradation products could be detected for the negative controls without enzyme, strongly indicating the degraded compounds are only released into the aqueous reaction medium upon enzymatic action. Similar products could be identified in the samples of the positive control using strong base (see FIGS. 8c, 9c and 10c).

Conclusions

The reported results demonstrate a profound degradation ability of the crude enzyme preparations from the metagenomic cutinase-like BC-CUT-013 (Biocatalysts Ltd., UK) towards the tested commercial polyurethane adhesives Adcote 102A and 545-75 as well as the coating Adcote 17-3. Interestingly, the metagenomic cutinase-like enzyme from BC-CUT-13 appears to be a more promiscuous enzyme, active on all the polyurethane materials especially Adcote 545-75 and Adcote 17-3. These superior degradation activities on commercial polyurethane materials have to our knowledge not been reported before in the prior art.

In contrast, the cutinases Thc_Cut1 and Thc_Cut2 as well as Thf_Cut (Biocatalyst Ltd., UK) showed overall lower degradation activity on all commercial polyurethane materials. Most materials tested so far for enzymatic degradation of polyurethanes in the prior art are more aliphatic. In this invention, the investigated commercial materials not only contain an aromatic fraction but also are comprised of a commercial formulation from DOW Chemicals with typical additives present which has not been reported elsewhere and thus, the ability of the enzyme BC-CUT-013 to depolymerize materials with a substantial aromatic fraction in the soft segment has, to our knowledge, not been described in the prior art.

The LC-HRMS analysis could clearly demonstrate that the results of the FDL assay do not only indicate probe release due to PU fragmentation and enhanced diffusion, but confirmed the degradation of the polymer matrix and revealed some of the polyurethanes structural composition.

Interestingly, the high degrees of degradation with BC-CUT-013 presented in FIGS. 1 and 2 were conducted with crude enzyme formulations using an enzyme loading corresponding to 5.6-7 μg protein per mg PU polymer and at ambient temperatures of 37° C. For example, the published study by Schmidt and colleagues (Schmidt et al. Polymers 2017, 9(2), 65) demonstrated only up to 4.9% and 4.1% weight loss for the commercial semi-aromatic polyester-polyurethane polymer Elastollan B85A-10 and C85A-10 BASF, respectively. Moreover, they used highly purified enzyme with an enzyme load of 0.63 μg enzyme per mg PU polymer at 70° C. within 8 days—much higher temperature conditions as presented in this invention. The inventors point out that the enzyme loading in this invention is based on total protein of which not all represents the active cutinase. In fact, the protein content of the herein used crude enzyme formulation only amounts to about 50%, or less, and thus the results regarding the actual cutinase loading are at least 50% lower demonstrating the excellent PU degradation efficiency of the BC-CUT-013 cutinase. Much higher activities can be expected when using purified enzyme.

Importantly, the adhesives and coatings used in this invention are typically found in packaging applications and thus their enzymatic degradation represents a realistic scenario. In contracts, the thermoplastic PU materials (TPU) used by Schmidt et al. (Polymers 2017, 9(2), 65) are not typically used as adhesives in packaging, but are rather applied and extruded into hoses, cable sheathing, belts, films and profiles, and can also be processed using blow molding and injection molding technologies (BASF).

The mild process conditions of this inventions and the possibility to apply and function with crude enzyme extracts are beneficial for a recycling process as they facilitate up-scaling, economic and environmental aspects.

### Example: 2: Optimal pH for Best Enzymes for Each Material

Material and Methods

Materials and Chemicals

The polyurethane materials Adcote 545-75 (75% w/w), Adcote 17-3 (75% w/w) and Co-reactant F (75% w/w) were thankfully provided by Dow Chemicals. Glycerol, K₂HPO₄, KH₂PO₄, fluorescein, fluorescein dilaurate, sodium hydroxide (NaOH) and ethyl acetate were all purchased from Sigma.

Based on analysis of degradation products by LC-HRMS following monomer contents could be confirmed (see Table 3). All materials contain phthalic acids as well as diethylene glycol. Adcote 17-3 also contains both sebacic acid as diacid component whereas Adcote 545-75 contains adipic acid. For the coating neo-pentyl-di-propanol could be detected. Co-reactant F was described in patents to contain isocyanate terminated polyol based branched pre-polymers. The isocyanate component was found to be toluene di-isocyanate (Wu et al, 2019, US20190284456A1).

TABLE 3
The table below lists the two materials tested (Adcote 545-75 and Adcote 17-3) their preparation and
identified components by LC-MS that have been released upon enzyme incubation and are thus
considered as building blocks (e.g., monomers). Information on co-reactant used for
creating the final two PU materials is also provided

| Name | Adcote 545-75 | Adcote 17-3 | Co-Reactant F |
|---|---|---|---|
| Description | Polyester component of two | solvent-based coating | Crosslinked, isocyanate |
| | component based adhesive | | containing component |
| Preparation | Cured with co-reactant F | Already cured | Cured with polyester |
| | | | component |
| Identified Components | 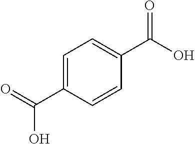 [SMILES: O=C(O)c1ccc(C(=O)O)cc1] | 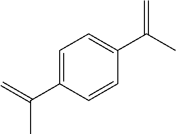 | 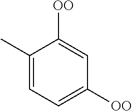 |
| | 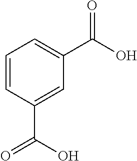 [SMILES: O=C(O)c1cccc(C(=O)O)c1] | 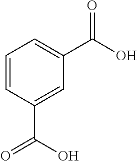 [SMILES: O=C(O)c1cccc(C(=O)O)c1] | 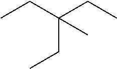 |
| | 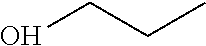 | 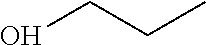 | 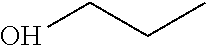 |
| | 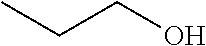 | 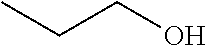 | 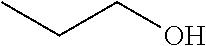 |
| | 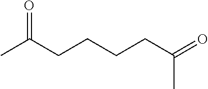 | 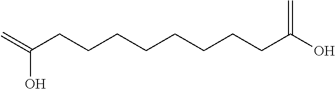 | |
| | | 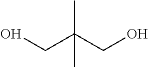 | |

The metagenomic cutinase BC-CUT-013 was purchased from Biocatalyst Ltd. UK. The enzyme BC-CUT-013 was identified via a metagenomic search against the query amino acid sequences from Thermobifida fusca CUT1.

TABLE 4
List of enzymes investigated, their type, abbreviation, organism
of origin, production organism, quality and supplier.

| | | | | Production | | Supplier/ |
|---|---|---|---|---|---|---|
| Name | Abbreviation | Type | Organism | Organism | Quality | Producer |
| Biocatalyst | BC-CUT-013 | Cutinase | Metagenomic | N/A | Crude | Biocatalyst |
| Cutinase 013 | | | | | extract | Ltd. UK |

The enzyme was diluted to a stock solution of 1 mg/ml protein in 40% (w/v) glycerol for easier handling during experiments.

Upon receiving of the polymer materials, 2.5× (polyester component) and 5× (co-reactant) stock solutions (w/w) were prepared by diluting the polymer in ethyl acetate. For the adhesive Adcote 575-75 the co-reactant had to be mixed with the polymer in ratios of 11.5:100 (w/w) respectively.

Preparation of Polyurethane Coated Glass Vials

The indirect fluorescent assay, established by Zumstein and colleagues (Zumstein, M. T., et al. (2017) Environmental Science & Technology 51(13): 7476-7485) is based on the assumption that the release of a homogeneous embedment reporter molecule (fluorescein dilaurate, FDL) in the target polymer matrix (adhesive or coating) is directly correlated to the degree of degradation of same polymer material. Only upon material degradation, FDL is released out of the polymer matrix and can then be hydrolyzed by an esterase-active enzyme into laureate and fluorescein, of which the latter molecule can be quantified fluorometrically (521/494 nm). One percent (%) polymer degradation is defined as one % release of originally embedded reporter molecule, in this case corresponding to 0.1 wt % incorporated FDL which was the optimum amount to reach a high detection limit while minimizing the effect on the polymer matrix and enzymes.

The stock solutions were used to prepare the casting solutions in ethyl acetate containing 12.6% (w/w) polymer and 0.0126% (w/w) FDL. This corresponds to a FDL:polymer ratio of 1:1000.

For 26.6 mg polymer, 200 μl of the casting solution were transferred into 2 ml HPLC vials (Agilent), vortexed for 2 s and then dried in a falcon rotator (Rotary-mixer 34526, Snijders) for 5-6 h. This was done to ensure an evenly spread coating on the inner side of the vial. As both Adcote 102A and 545-75 constitute glues this ensured a clean even surface for the reactions.

The vials were then dried at 50° C. for 1 h before leaving them to cure for 1 week at room temperature.

Single Enzyme Activity Screening of BC-CUT-013 at pH 6.5-8 on Adcote 17-3 and 545-75

The reaction was carried out in 1.5 ml 0.1 potassium-saline phosphate-Buffer (pH 6.5-8) with an enzyme load of 5.64-7 μg protein/mg polymer. This was done to ensure pH stability as acids are formed upon hydrolysis that may affect the enzyme negatively. The buffer was prepared by mixing K₂HPO₄ and KH₂PO₄ according to the Henderson-Hasselbalch equation.

As a positive control reaction for the assay, the FDL-loaded polymer sample was exposed to a 1M sodium hydroxide (NaOH) solution as stability of long-chain fluorescein diesters and greatly decreases above pH 8.5 (Guilbault, G. G., & Kramer, D. N. (1966), 14(1), 28-40). In addition, ester bonds as in polyesters as well as urethanes bonds present in polyurethanes can be hydrolysed at elevated pH as reported in a study by Matuszak and colleagues (Matuszak, M. L., Frisch, K. C., & Reegen, S. L. (1973), Journal of Polymer Science: Polymer Chemistry Edition, 11(7), 1683-1690). Thus, a basic solution of 1M NaOH is used as positive control for the indirect FDL assay.

As a negative control, FDL-loaded polymer sample were exposed to the respective buffer solution without enzyme or NaOH. Leakage of FDL was determined negligible.

All vials were incubated at 37° C. at 250 rpm. Samples of 50 μl were taken after 0, 1, 2, 3, 4, 5, 6, 14 days, mixed with 150 μl 4M NaOH and measured at 494/521 nm in a plate reader. This was done to ensure full hydrolysis of all free released FDL. A fluorescein calibration curve of 3.125-5 μM was used to calculate the FDL release.

Results and Discussion

As described in the literature, the pH of the reaction solution can have a profound effect on the activity of the enzyme and previous studies have shown a higher pH above 7.5 can be more beneficial for polyester degradation, e.g. PET (see Furukawa, M. et al. 2018, ChemSusChem, 11(23), 4018-4025).

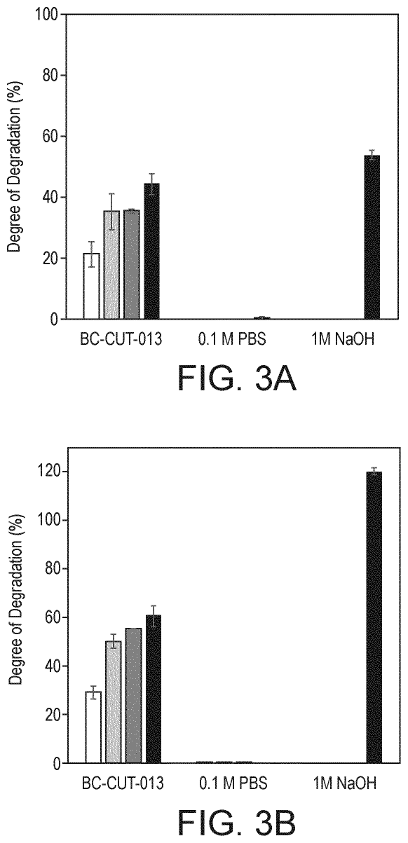

Indeed, changes in pH (6.5, 7, 7.5 and 8) had also a significant effect on the ability of BC-CUT-013 to degrade PU materials (Adcote 545-75 and Adcote 17-3), see FIGS. 3A-B and 4A-B.

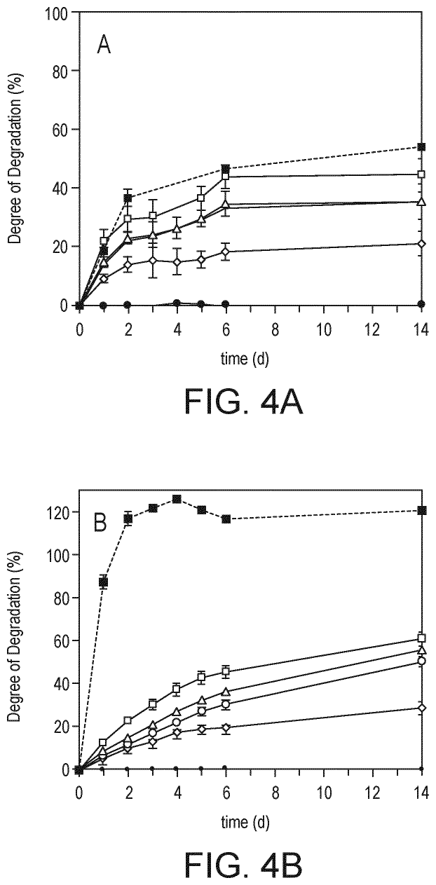

Interestingly, as shown in FIGS. 3 and 4, the enzyme BC-CUT-013 showed highest activity at a basic pH of around 8.2 for both the adhesive Adcote 545-75 and the coating Adcote 17-3, while other enzymes had different pH optima depending on the substrate used (data not shown). It is thus not always trivial which pH provides the optimal conditions for degradation of polymers. The degree of degradation was more than doubled for BC-CUT-013, from 20% to 61% and 29% to 61% for Adcote 545-75 and Adcote 17-3, respectively, when increasing the reaction pH from 6.5 to 8 (FIG. 4).

The efficiency of the delamination process could be greatly enhanced by choosing the optimal pH for the reaction, as the presented results demonstrate. The invention provides the optimal pH ranges for the especially active cutinase BC-CUT-013 on Adcote 545-75 and Adcote 17-3, which will aid the process development for performing delamination of multilayer laminates.

The here presented results on single enzyme activity for commercial PU degradation are unique. Firstly, due to the commercial application of the tested polyurethane polymers in food packaging, secondly, the high degree of degradation up to 61% under mild reaction conditions (37° C. and pH 6.5-8) and finally because of the high activity of commercial and crude enzyme loadings of 5.6-7 protein μg/mg polymer which all have not been described for polyurethanes before in the prior art.

### Example 3: Enzymatic Degradation Adcote 17-3 Coating by Combination of Two Cutinases (Thc_Cut1 and BC-CUT-013)

Materials and Methods

Materials and Chemicals

The polyurethane material Adcote 17-3 (75% w/w) was thankfully provided by Dow Chemicals. Glycerol, K₂HPO₄, KH₂PO₄, Flourescein, Flourescein dilaurate, NaOH and Ethylacetate were all purchased from Sigma.

Based on analysis of degradation products by LC-HRMS following monomer contents could be confirmed (see Table 5). The material contained phthalic acids as well as diethylene glycol, but also contain both sebacic acid as diacid component whereas neo-pentyl-dipropanol could also be detected.

TABLE 5
Material description for Adcote 17-3, PU coating, its preparation and
identified components by LC-HRMS that have been released upon
enzymatic treatment and are thus considered building
blocks (i.e., monomers).

| Name | Adcote 17-3 |
|---|---|
| Description | solvent-based coating |
| Preparation | Already cured |
| Identified Components | 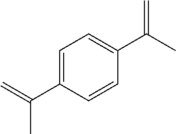 |
| | 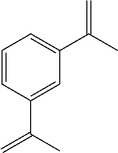 |
| | 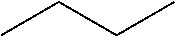 |
| | 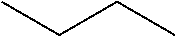 |
| | 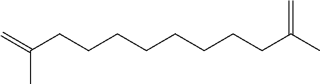 |
| | 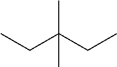 |

Thc_Cut1 (T. cellulosilytica 1), as well as the metagenomic cutinase BC-CUT-013 were purchased from Biocatalyst Ltd. UK. All of these enzymes used were used as crude extract, non-purified, which represents a more industrially relevant and cheaper preparation than purified enzymes that are too costly for such proposed waste application.

TABLE 6
List of enzymes investigated, their type, abbreviation, organism
of origin, production organism, quality and supplier.

| | | | | Production | | Supplier/ |
|---|---|---|---|---|---|---|
| Name | Abbreviation | Type | Organism | Organism | Quality | Producer |
| T. cellulosilytica 1 | Thc_Cut1 | Cutinase | T. cellulosilytica | Recombinant | Crude | Biocatalysts |
| Biocatalyst | BC-CUT-013 | Cutinase | Metagenomic | E. coli | soluble | Ltd. UK |
| Cutinase 013 | | | | | extract | |

All enzymes were diluted to stock solutions of 1 mg/ml protein in 40% (w/v) glycerol for easier handling during experiments.

Upon receiving of the polyurethane materials, 2.5× solutions (w/w) were prepared by diluting the polymer in ethyl acetate.

Preparation of Polyurethane Coated 96 Well Plates

The stock solutions were used to prepare the casting solutions in ethyl acetate containing 2.3% (w/w) polymer and 0.0023% (w/w) FDL. This corresponds to a FDL:polymer ratio of 1:1000.

The indirect fluorescent assay, established by Zumstein and colleagues (Zumstein, M. T., et al. (2017) Environmental Science & Technology 51(13): 7476-7485) is based on the assumption that the release of a homogeneous embedment reporter molecule (fluorescein dilaurate, FDL) in the target polymer matrix (adhesive or coating) is directly correlated to the degree of degradation of same polymer material. Only upon material degradation, FDL is released out of the polymer matrix and can then be hydrolyzed by an esterase-active enzyme into laureate and fluorescein, of which the latter molecule can be quantified fluorometrically (521/494 nm). One percent (%) polymer degradation is defined as one % release of originally embedded reporter molecule, in this case corresponding to 0.1 wt % incorporated FDL which was the optimum amount to reach a high detection limit while minimizing the effect on the polymer matrix and enzymes.

For 0.79 mg polymer per well, 40 μl of the casting solution were transferred to solvent-resistant 96-well plates (Greiner 655219, Greiner Bio-One), before leaving them to cure for 1 week at room temperature.

Enzyme Activity Screening of the Enzyme Combination Thc_Cut1 and BC-CUT-013 on Adcote 17-3

The reaction was carried out in 200 μl 0.1 potassium-saline phosphate-Buffer (pH 7) with 25.6 μg protein/mg polymer enzyme load for a single enzyme reaction and for each enzyme respectively in for the 1:1 combination (in total 51.2 μg protein/mg polymer). The buffer was chosen to ensure pH stability as acids are formed upon hydrolysis that may affect the enzyme negatively. The buffer was prepared by mixing K₂HPO₄ and KH₂PO₄ according to the Henderson-Hasselbalch equation.

As a positive control reaction for the assay, the FDL-loaded polymer sample was exposed to a 1M sodium hydroxide (NaOH) solution as stability of long-chain fluorescein diesters and greatly decreases above pH 8.5 (Guilbault, G. G., & Kramer, D. N. (1966), 14(1), 28-40). In addition, ester bonds as in polyesters as well as urethanes bonds present in polyurethanes can be hydrolysed at elevated pH as reported in a study by Matuszak and colleagues (Matuszak, M. L., Frisch, K. C., & Reegen, S. L. (1973), Journal of Polymer Science: Polymer Chemistry Edition, 11(7), 1683-1690). Thus, a basic solution of 1M NaOH is used as positive control for the indirect FDL assay.

As a negative control, FDL-loaded polymer sample were exposed to the respective buffer solution without enzyme or NaOH. Leakage of FDL was determined negligible. All plates were incubated at 37° C. at 250 rpm and measured after 0, 2, 4, 6, 8, 10 and 24 h at 494/521 nm in a plate reader.

A fluorescein calibration curve of 0.03125-5 μM was used to calculate the FDL release. After 24 h the reaction the reaction was stopped and all plates stored at −20° C.

Results and Discussion

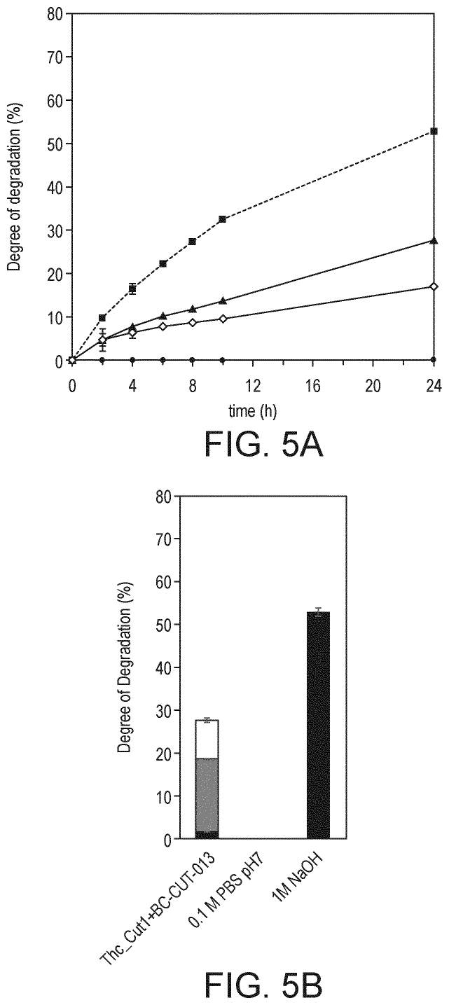

The inventors were surprised that the degradation yield could be greatly improved when combining two cutinases. As shown in FIGS. 5A-B, the degradation efficiency was enhanced from 17% to 27% when combining Thc_cut1 and BC-CUT-013 in a ratio of 1:1 (mg/mg) on the PU coating Adcote 17-3. Typically, the combination of different enzyme types have been reported to provide an increase in the degradation efficiency of complex substrates like cellulose (e.g., cellulases and monooxygenases) and few studies on polyesters (Barth, M. et al. 2015. Biochemical Engineering Journal, 93, 222-228) and polyurethanes. Polyurethanes have been subjected to enzyme cocktails by combining different types of enzyme, such as, esterase and amidase (Magnin, A., et al. 2019. Waste Management, 85, 141-150) or esterases and a protease (Ozsagiroglu, et al. 2012. Polish Journal of Environmental Studies 21.6: 1777-1782), of which however, former could only detect the release of few building blocks but no higher mass release and latter only found a competitive (negative) effect. Hence, this invention provides a new enzyme combination on PU based coating that drastically enhances the degradation of PU-based polymers through a synergistic effect.

The inventors hypothesize that the drastic degradation gain by using two cutinases (Thc-Cut1 and BC-CUT-013) in this invention is based on a synergistic effect, for example, of complementary substrate specificity that allows the elimination of inhibitory degradation products by one enzyme to enhance the activity of the other, or the enzyme combination introduces an endo- and an exo-activity, or complementary cleavage sites that enable a more broad hydrolysis at various polymer locations thereby leading to a faster and more comprehensive PU degradation.

The inventors point out that the degradation gain of 1.6-fold as shown in FIG. 5B compares to the sum of degree of degradation for the two individual enzymes and thus depicts the real synergistic gain of the enzyme combination. Notably, combinations of other enzymes, for example, were found to exhibit no or even negative effects and thus the single degradation activity was the same or higher than in combination (data not shown).

The application of enzyme combination could be used in a more efficient and faster decoating process of multi-layered materials.

### Example 4: Enzymatic Degradation of 30% Recycled PET by Four Cutinases

Materials and Methods

Materials and Chemicals

The polyethylene terephthalate (PET) used for the enzymatic assays was post-consumer PET from Henniez still water bottles of 33 cL, with 30% recycled PET (rPET). Glycerol, K₂HPO₄, KH₂PO₄, NaOH and ethylacetate, hydrochloric acid, formic acid, and methanol were all purchased from Sigma. Terephthalic acid (TPA) was purchased from Fisher Scientific, dimethyl sulfoxide (DMSO) was from Fluka.

Thf_Cut1 (T. fusca), Thc_Cut2 (T. cellulosilytica) and ThcCut1 (T. cellulosilytica) as well as the metagenomic cutinases BC-CUT-013 was purchased from Biocatalyst.

TABLE 7
List of enzymes investigated, their type, abbreviation, organism
of origin, production organism, quality and supplier.

| | | | | Production | | Supplier/ |
|---|---|---|---|---|---|---|
| Name | Abbreviation | Type | Organism | Organism | Quality | Producer |
| T. fusca cutinase | Thf_Cut | Cutinase | T. Fusca | Recombinant | Crude | Biocatalysts |
| T. cellulosilytica | Thc_Cut2 | Cutinase | T. cellulosilytica | E. coli | soluble | Ltd. UK |
| cutinase 2 | | | | | extract | |
| T. cellulosilytica 1 | Thc_Cut1 | Cutinase | T. cellulosilytica | | | |
| Biocatalyst | BC-CUT-013 | Cutinase | Metagenomic | | | |
| Cutinase 013 | | | | | | |

All enzymes were diluted to stock solutions of 1 mg/mi protein in 40% (w/v) glycerol for easier handling during experiments.

Enzymatic Hydrolysis of Post-Consumerpolyethylene Terephthalate (PET)

The post-consumer water bottles were pre-treated before being submitted to enzymatic treatment. The PET was cut in squares of 1-2 cm, washed with Ethanol (for about 30 mi) and dried at 37° C. The PET was subsequently shredded using a 6870D Freezer/Mill® Cryogenic Grinder from SPEX® SamplePrep. The shredded PET was sieved, separating pieces into four size categories: <0.2 mm, 0.2-0.5 mm, 0.5-1 mm, and >1 mm.

Approximately 20-25 mg of pre-treated post-consumer PET powder was placed into 4 mL glass vials with a PTFE/silicone/PTFE septum. The reactions were carried out at 37° C. in 1.5 mL freshly prepared enzyme solutions in 100 mM Na₂HPO₄/NaH₂PO₄ buffer at pH 7. The final enzyme load corresponded to 6-7.5 μg/mg polymer.

The reactions were performed on the horizontal in an ISF1-X incubator shaker from Kuhner Shaker at 100 rpm, to keep the PET particles in suspension. Control reactions were performed with buffer instead of enzyme solution. Samples were taken after every 24 h. At the end of the reactions, the PET was washed two times with MilliQ and one time with ethanol, dried at room temperature and stored for further analysis.

HPLC

The products of the enzymatic hydrolysis of PET were quantified by high-pressure liquid chromatography (HPLC). Samples of 50 μL were taken and transferred to tubes on ice containing 205 μL of 25 mM HCl in the HPLC mobile phase (0.1% formic acid in 30% MeOH), to stop the reaction and precipitate the enzymes. The samples were then centrifuged at 16000 g at 0° C., for 15 min. Approximately 200 μL of the supernatant was transferred to HPLC glass vials. The samples were analyzed by reversed phase chromatography using an Agilent 1200 series system, equipped with an Acquity UPLC HSS C18 1.8 μm 2.1×50 mm column from Waters and a diode array detector (DAD), with detection at 241 nm. A volume of 5 or 10 μL sample was injected into the system. The flow was 0.2 mL/min, the column operated at 50° C. and the run time was 8 min. Calibration standards of terephthalic acid (TPA), mono(2-hydroxyethyl terephthalate) (MHET) and bis(2-hydroxyethyl terephthalate) (BHET) were prepared in the same way as samples, with concentrations ranging from 0.005 to 1 mM. Stock solutions of 10 mM of all compounds were prepared in DMSO.

Results and Discussion

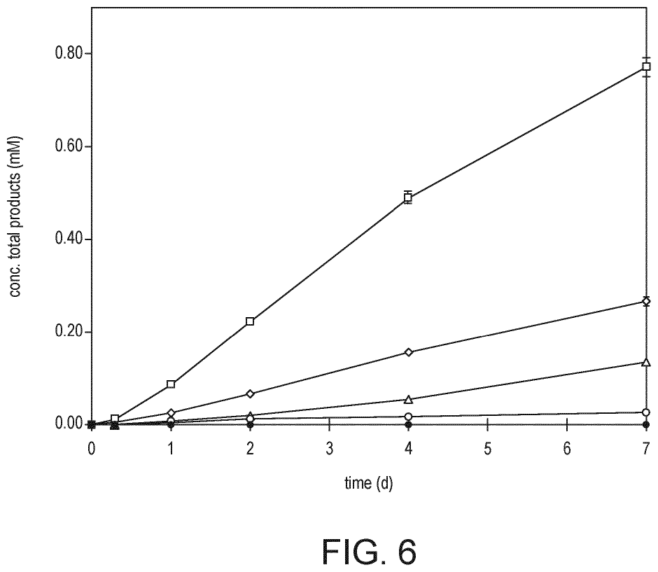

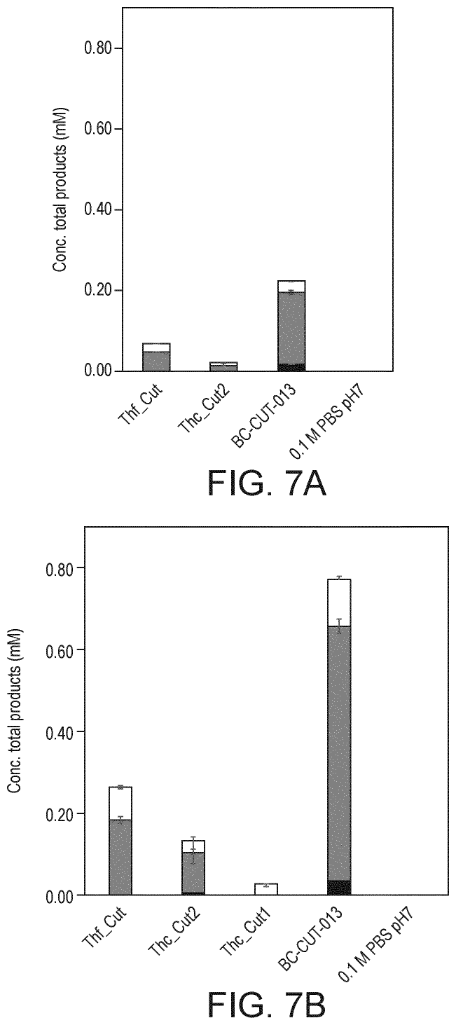

As shown in FIGS. 6 and 7, of all tested enzymes, the metagenomic cutinase BC-CUT-013 demonstrated highest total product formation during the degradation of PET within 7 days at pH 7 and 37° C. exceeding the three widely reported PET degrading enzymes Thf_cut, Thc_cut2 and Thc_cut1 by a factor of three with 0.76 mM after 7 days (see FIG. 7B).

The use of recycled PET (rPET) in this invention appears to impact the degradation efficiency as the previously reported degradation rates of Thf_cut, Thc_cut2 and Thc_cut1 (e.g., Schmidt, et al. 2017. Polymers, 9(2), 65.) were lower and could not be reproduced. Yet, this also demonstrates the higher efficiency of BC-CUT-013. Furthermore, reactions reported in the literature were typically performed at much higher temperatures (e.g. 50° C. or 70°) as compared to the lower heat energy input of 37° C. used herein. This invention represents to our knowledge the first report on the use of rPET samples with a recycled content of 30%.

Importantly, the cutinase BC-CUT-013 demonstrated high activity on both the polyester-containing polyurethane adhesives as well as rPET. Consequently, this enzyme (BC-CUT-013) or the combination of both BC-CUT-013 and Thc_cut2 could be applied in a process where PET and adhesives can be degraded sequentially or simultaneously in a process targeted to delaminate, e.g., a multilayer structure with a PE base layer and this cleaned PE could then be fed into the PE recycling stream.

1. Method of degrading non-biodegradable polyurethane (PU) in packaging material comprising the step of subjecting the packaging material comprising PU deprived of urea linkages to at least one cutinase, wherein said at least one cutinase is selected from the group consisting of BC-CUT-013, Thc_Cut1, and combinations thereof, and is used as a crude extract.

2. Method in accordance with claim 1, wherein the at least one cutinase is used in a concentration of at least about 0.06 μg protein/mg polymer.

3. Method in accordance with claim 1, wherein the PU is subjected to the at least one cutinase at a temperature in the range of 20-50° C.

4. Method in accordance with claim 1, wherein the PU is subjected to the at least one cutinase at a pH in the range of about 6-9.

5. Method in accordance with claim 1, wherein the PU is subjected to the at least one cutinase for at least 3 days.

6. Method in accordance with claim 1, wherein the PU is present in food packaging.

7. Method in accordance with claim 6, wherein the packaging comprises a multilayer packaging structure comprising at least two polymeric layers, wherein the polymeric layers comprises a PU-based layer and at least one layer selected from the group consisting of a further PU-based layer, a polyethylene terephthalate (PET)-based layer, a polyethylene (PE)-based layer, and a combination thereof.

8. Method in accordance with claim 6, wherein the PU is present in the packaging in a PU-based adhesive or a PU based-coating.

9. Method in accordance with claim 1, wherein the method is used for the selective delamination of at least one PU-based layer in a multilayer packaging.

10. Method in accordance with claim 1, wherein the PU is present in a packaging comprising a multilayer packaging structure, wherein the multilayer packaging structure comprises a base layer that can be recycled my and at least one PU-based layer, wherein the method is used to recycle the multilayer packaging structure by degrading the at least one PU-based layer and by subjecting the base layer to a recycling stream.

11. Method in accordance with claim 1, wherein the method further comprises the step of reducing the particle size of the PU and/or the PU containing material before or during subjecting the PU and the PU containing material to at least one cutinase.

12. Method in accordance with claim 11, wherein the particle size is reduced by a mechanical treatment to particles with an average diameter of less than about 5 mm.

13. Method in accordance with claim 1, wherein the method is carried out in a closed vessel.
